(19) United States
(12) Patent Application Publication
(10) Pub. No.: US 2026/0124354 A1
(43) Pub. Date: May 7, 2026

(54) SYSTEMS AND METHODS FOR NEGATIVE PRESSURE WOUND THERAPY SYSTEM WITH NOISE CANCELLING

(71) Applicant: KCI Manufacturing Unlimited Company, Westmeath (IE)

(72) Inventors: Benjamin A. PRATT, Poole (GB); James SEDDON, Wimborne (GB); Timothy M. ROBINSON, Shillingstone (GB)

(21) Appl. No.: 18/706,324

(22) PCT Filed: Sep. 26, 2022
(86) PCT No.: PCT/IB2022/059119

§ 371 Date: Apr. 30, 2024

Related U.S. Application Data
(60) Provisional application No. 63/275,033, filed on Nov. 3, 2021.

(51) Int. Cl. A61M 1/00

(52) U.S. Cl. CPC A61M 1/966; A61M 1/962; A61M 2205/3375; A61M 2205/42

(57) ABSTRACT

A negative pressure wound therapy (NPWT) device includes a pneumatic pump, a single microphone, and a controller. The pneumatic pump is operable to draw a negative pressure at a wound dressing. The single microphone is configured to measure a sound level of an environment surrounding the NPWT device. The controller is configured to operate the pneumatic pump to draw the negative pressure at the wound dressing. The controller is further configured to obtain the sound level of the environment surrounding the NPWT device, and adjust operation of the pneumatic pump based on the sound level of the environment.

### CROSS-REFERENCE TO RELATED APPLICATIONS

This application is a U.S. National Stage Entry of PCT/IB2022/059119, filed Sep. 26, 2022, which claims the benefit, under 35 U.S.C. § 119 (c), of U.S. Provisional Application No. 63/275,033, filed on Nov. 3, 2021, each of which are incorporated herein by reference in its entirety.

### BACKGROUND

The present disclosure relates generally to a wound therapy system, and more particularly to a negative pressure wound therapy system.

### SUMMARY

One implementation of the present disclosure is a negative pressure wound therapy (NPWT) device, according to some embodiments. In some embodiments, the NPWT device includes a pneumatic pump, a single microphone, and a controller. In some embodiments, the pneumatic pump is operable to draw a negative pressure at a wound dressing. In some embodiments, the single microphone is configured to measure a sound level of an environment surrounding the NPWT device. In some embodiments, the controller is configured to operate the pneumatic pump to draw the negative pressure at the wound dressing. In some embodiments, the controller is further configured to obtain the sound level of the environment surrounding the NPWT device, and adjust operation of the pneumatic pump based on the sound level of the environment.

In some embodiments, the controller is configured to adjust operation of the pneumatic pump to reduce sound emitted by the pneumatic pump in response to the sound level of the environment exceeding a threshold. In some embodiments, adjusting operation of the pneumatic pump includes adjusting a duty cycle of the pneumatic pump.

In some embodiments, the NPWT device further includes a pressure sensor. In some embodiments, the controller is configured to determine if the negative pressure is maintained at the wound dressing based on information received from the pressure sensor.

In some embodiments, the controller is configured to, in response to the negative pressure not being maintained at the wound dressing: determine if the negative pressure is at a lowest acceptable value, and in response to the negative pressure not being at the lowest acceptable value, reduce a magnitude of the negative pressure by adjusting operation of the pump, and obtain the sound level of the environment surrounding the NPWT device. In some embodiments, in response to the negative pressure not being maintained and the negative pressure being above the lowest acceptable value, the controller is configured to operate a display of the NPWT device to provide a leak alarm, and operate the pneumatic pump to maintain the negative pressure at the lowest acceptable value.

In some embodiments, the controller is configured to, in response to the negative pressure being maintained at the wound dressing, obtain the sound level of the environment surrounding the NPWT device, and adjust operation of the pneumatic pump based on the sound level of the environment to maintain the sound level of the environment below a threshold.

In some embodiments, the pneumatic pump is positioned within a cavity of the NPWT device. In some embodiments, the cavity includes sound absorbing material and an opening. In some embodiments, the sound absorbing material is configured to absorb sound emitted by the pneumatic pump as the pneumatic pump operates, and the pneumatic pump is configured to emit sound through the opening to the environment. In some embodiments, the controller is configured to operate the pneumatic pump to provide the negative pressure at a reduced magnitude in response to the sound level of the environment exceeding a threshold and a pump duty cycle of the pneumatic pump being at a lowest acceptable value.

Another implementation of the present disclosure is a NPWT device, according to some embodiments. In some embodiments, the NPWT device includes a pneumatic pump, a single microphone, a single speaker, and a controller. In some embodiments, the pneumatic pump is operable to draw a negative pressure at a wound dressing. In some embodiments, the single microphone is configured to measure a sound level of an environment surrounding the NPWT device. In some embodiments, the single speaker is configured to emit a sound for noise cancellation. In some embodiments, the controller is configured to obtain the sound level of the environment surrounding the NPWT device as the pneumatic pump operates to draw the negative pressure. In some embodiments, the controller is also configured to adjust a duty cycle of the pneumatic pump in response to the sound level of the environment exceeding a threshold and obtain the sound level of the environment surrounding the NPWT device as the pneumatic pump operates according to the adjusted duty cycle. In some embodiments, in response to the sound level of the environment surrounding the NPWT device exceeding the threshold as the pneumatic pump operates according to the adjusted duty cycle, the controller is configured to operate the single speaker to provide a sound wave having a frequency to provide destructive interference to sound waves emitted into the environment by the pneumatic pump to reduce the sound level of the environment.

In some embodiments, the controller is configured to select a profile for the speaker from multiple predetermined profiles based on at least one of the adjusted duty cycle of the pneumatic pump or the sound level of the environment, and operate the speaker according to the profile to emit the sound wave for noise cancellation.

In some embodiments, the single microphone is configured to measure both a sound level of the environment, and a characteristic of sound waves in the environment. In some embodiments, the controller is configured to determine the frequency for the sound wave provided by the single speaker based on the characteristic of the sound wave in the environment.

In some embodiments, the characteristic includes at least one of an amplitude or a frequency of sound waves in the environment of the NPWT device. In some embodiments, the single speaker is positioned proximate a location on the NPWT device where sound waves emitted by the pneumatic device are propagated into the environment of the NPWT device.

In some embodiments, the pneumatic pump is positioned within a cavity of the NPWT device. In some embodiments, the cavity includes sound absorbing material and an opening. In some embodiments, the sound absorbing material is configured to absorb sound emitted by the pneumatic pump as the pneumatic pump operates, and the pneumatic pump is configured to emit sound through the opening to the environment. In some embodiments, the opening is the location of the NPWT device where sound waves emitted by the pneumatic device are propagated into the environment of the NPWT device.

In some embodiments, the pneumatic pump is positioned within a cavity of the NPWT device. In some embodiments, the cavity includes sound absorbing material and an opening. In some embodiments, the sound absorbing material is configured to absorb sound emitted by the pneumatic pump as the pneumatic pump operates, and the pneumatic pump is configured to emit sound through the opening to the environment.

In some embodiments, the controller is configured to adjust operation of the pneumatic pump to provide the negative pressure having a reduced magnitude in response to the sound level of the environment exceeding the threshold amount as the single speaker is operated to provide the sound wave.

Another implementation of the present disclosure is a NPWT device, according to some embodiments. In some embodiments, the NPWT device includes a pneumatic pump, at least one microphone, multiple speakers, and a controller. In some embodiments, the pneumatic pump is operable to draw a negative pressure at a wound dressing. In some embodiments, the microphones are configured to measure a sound level of an environment surrounding the NPWT device. In some embodiments, the multiple speakers are configured to emit sound for noise cancellation in multiple directions. In some embodiments, the controller is configured to obtain the sound level of the environment surrounding the NPWT device as the pneumatic pump operates to draw the negative pressure. In some embodiments, the controller is also configured to adjust a duty cycle of the pneumatic pump in response to the sound level of the environment exceeding a threshold, and obtain the sound level of the environment surrounding the NPWT device as the pneumatic pump operates according to the adjusted duty cycle. In some embodiments, in response to the sound level of the environment surrounding the NPWT device exceeding the threshold as the pneumatic pump operates according to the adjusted duty cycle, the controller is configured to operate the multiple speakers to provide sound waves in the multiple directions, the sound waves having frequencies to provide destructive interference to sound waves emitted into the environment by the pneumatic pump to reduce the sound level of the environment.

In some embodiments, the controller is configured to select a profile for each of the multiple speakers from multiple predetermined profiles based on at least one of the adjusted duty cycle of the pneumatic pump or the sound level of the environment. In some embodiments, the controller is also configured to operate the multiple speakers according to each of the profiles to emit the sound waves for noise cancellation.

In some embodiments, the single microphone is configured to measure both a sound level of the environment, and a characteristic of sound waves in the environment. In some embodiments, the controller is configured to determine the frequency for the sound wave provided by the single speaker based on the characteristic of the sound wave in the environment.

In some embodiments, the characteristic includes at least one of an amplitude or a frequency of sound waves in the environment of the NPWT device. In some embodiments, at least one of the multiple speakers are positioned proximate a location on the NPWT device where sound waves emitted by the pneumatic device are propagated into the environment of the NPWT device.

In some embodiments, the pneumatic pump is positioned within a cavity of the NPWT device. In some embodiments, the cavity includes sound absorbing material and an opening. In some embodiments, the sound absorbing material is configured to absorb sound emitted by the pneumatic pump as the pneumatic pump operates, and the pneumatic pump is configured to emit sound through the opening to the environment. In some embodiments, the opening is the location of the NPWT device where sound waves emitted by the pneumatic device are propagated into the environment of the NPWT device.

In some embodiments, the pneumatic pump is positioned within a cavity of the NPWT device. In some embodiments, the cavity includes sound absorbing material and an opening. In some embodiments, the sound absorbing material is configured to absorb sound emitted by the pneumatic pump as the pneumatic pump operates, and the pneumatic pump is configured to emit sound through the opening to the environment.

In some embodiments, the controller is configured to adjust operation of the pneumatic pump to provide the negative pressure having a reduced magnitude in response to the sound level of the environment exceeding the threshold amount as the single speaker is operated to provide the sound wave.

Another implementation of the present disclosure is a NPWT device, according to some embodiments. In some embodiments, the NPWT device includes a pneumatic pump, multiple microphones, multiple speakers, and a controller. In some embodiments, the pneumatic pump is operable to draw a negative pressure at a wound dressing. In some embodiments, the multiple microphones are each configured to measure a sound level and a characteristic of an environment surrounding the NPWT device in a separate direction. In some embodiments, the multiple speakers are each configured to emit sound for noise cancellation in the separate directions. In some embodiments, the controller is configured to obtain the sound levels and the characteristics of the environment in each of the different directions as the pneumatic pump operates to draw the negative pressure. In some embodiments, in response to any of the sound levels of the environment surrounding the NPWT device exceeding a threshold as the pneumatic pump operates, the controller is configured to operate at least one of the speakers to provide sound waves in the directions. In some embodiments, the sound waves have frequencies to provide destructive interference to sound waves emitted into the environment by the pneumatic pump to reduce the sound level of the environment in any of the directions.

In some embodiments, the controller is configured to determine the frequencies of the sound waves emitted by the speakers in each of the separate directions based on a corresponding one of the characteristics measured by one of the plurality of microphones.

In some embodiments, the controller is configured to operate each of the speakers independently in real-time based on the characteristics and sound level of sound in the different directions to provide active noise cancelling in each of the different directions. In some embodiments, each of the speakers are positioned proximate a corresponding one of the microphones.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a block diagram of a wound therapy system including a therapy device coupled to a wound dressing via tubing, according to some embodiments.

FIG. 2 is a block diagram illustrating the therapy device of FIG. 1 in greater detail when the therapy device operates to draw a vacuum within a negative pressure circuit, according to some embodiments.

FIG. 3A is a block diagram illustrating the therapy device of FIG. 1 in greater detail when the therapy device operates to vent the negative pressure circuit, according to some embodiments.

FIG. 3B is a block diagram illustrating the therapy device of FIG. 1 in greater detail when the therapy device uses an orifice to vent the negative pressure circuit, according to some embodiments.

FIG. 4 is a block diagram illustrating the therapy device of FIG. 1 in greater detail when the therapy device operates to deliver instillation fluid to the wound dressing and/or a wound, according to some embodiments.

FIG. 5 is a perspective view of a therapy device including a microphone and a pneumatic pump, illustrating sound emitted due to operation of the therapy device, according to some embodiments.

FIG. 6 is a block diagram of the therapy device of FIG. 5, according to some embodiments.

FIG. 7 is a top view of the therapy device of FIG. 5 illustrating sound waves emitted when the pneumatic pump of the therapy device operates according to a first duty cycle, according to some embodiments.

FIG. 8 is a top view of the therapy device of FIG. 5 illustrating sound waves emitted when the pneumatic pump of the therapy device operates according to a second duty cycle, according to some embodiments.

FIG. 9 is a flow diagram of a process for operating the therapy device of FIGS. 5-8 to maintain negative pressure at a dressing and reduce sound output, according to some embodiments.

FIG. 10 is a perspective view of a therapy device including a microphone, a pneumatic pump, and a single speaker, illustrating sound emitted due to operation of the therapy device and sound emitted by the single speaker for noise cancellation, according to some embodiments.

FIG. 11 is a block diagram of the therapy device of FIG. 10, according to some embodiments.

FIG. 12 is a flow diagram of a process for operating the therapy device of FIGS. 10-11 to provide noise cancellation, according to some embodiments.

FIG. 13 is a diagram of steps performed to calibrate the therapy device of FIGS. 10-11, according to some embodiments.

FIG. 14 is a side view of a therapy device including a chamber having sound absorbing material within which a pneumatic pump is positioned, according to some embodiments.

FIG. 15 is a perspective view of a therapy device including a single microphone, a single speaker, and a pneumatic pump positioned within a chamber, according to some embodiments.

FIG. 16 is a diagram of steps performed to calibrate the therapy device of FIG. 15, according to some embodiments.

FIG. 17 is a perspective view of a therapy device including a single microphone, multiple speakers, and a pneumatic pump, illustrating sound emitted due to operation of the therapy device and sound emitted by the multiple speakers for noise cancellation, according to some embodiments.

FIG. 18 is a diagram of steps performed to calibrate the therapy device of FIG. 17, according to some embodiments.

FIG. 19 is a perspective view of a therapy device including multiple microphones, multiple speakers, and a pneumatic pump, illustrating sound emitted due to operation of the therapy device and sound emitted by the multiple speakers for noise cancellation, according to some embodiments.

FIG. 20 is a flow diagram of a process for controlling the therapy device of FIG. 19, according to some embodiments.

FIG. 21 is a block diagram of the therapy device of FIG. 19, according to some embodiments.

### DETAILED DESCRIPTION

### Overview

Referring generally to the FIGURES, systems and methods for reducing noise emitted by a NPWT device are shown. The NPWT device may include a pneumatic pump configured to draw a negative pressure at a dressing for NPWT. The pneumatic pump may emit noises during operation. The NPWT device can reduce a sound level of noise emitted by the pneumatic pump during operation by adjusting a duty cycle of the pneumatic pump, operating a single speaker according to various predefined profiles to provide noise cancellation, operating multiple speakers according to various predefined profiles to provide noise cancellation, or operating multiple speakers based on sensor or microphone date to provide active noise cancellation. In some embodiments, the pneumatic pump may be positioned within a sound absorbing chamber of the NPWT device to further limit or reduce sound output.

### Wound Therapy System

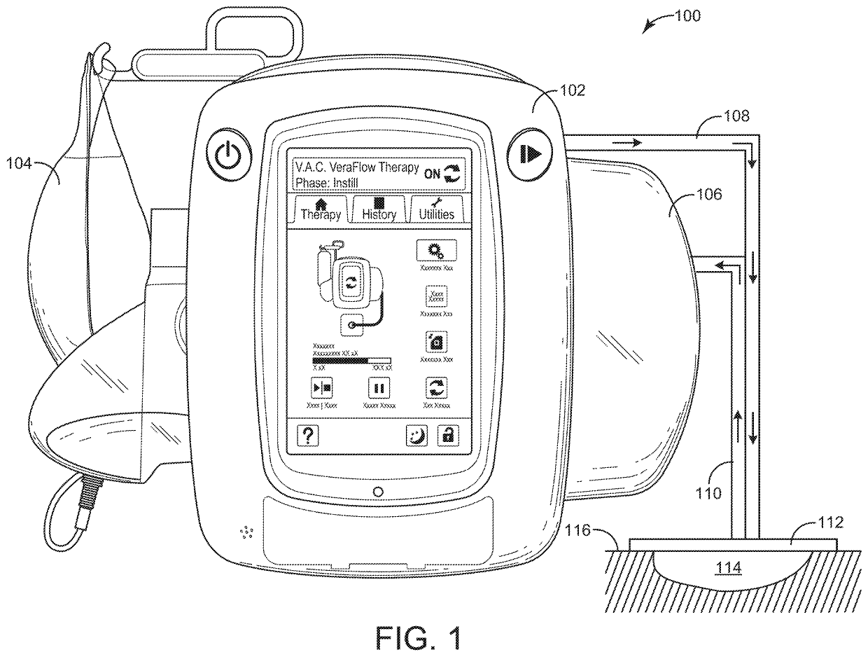

Referring now to FIGS. 1-4, a NPWT system 100 is shown, according to an exemplary embodiment. NPWT system 100 is shown to include a therapy device 102 fluidly connected to a wound dressing 112 via tubing 108 and 110. Wound dressing 112 may be adhered or sealed to a patient's skin 116 surrounding a wound 114. Several examples of wound dressings 112 which can be used in combination with NPWT system 100 are described in detail in U.S. Pat. No. 7,651,484 granted Jan. 26, 2010, U.S. Pat. No. 8,394,081 granted Mar. 12, 2013, and U.S. Pat. No. 10,232,155 granted Mar. 19, 2019. The entire disclosure of each of these patents is incorporated by reference herein.

Therapy device 102 can be configured to provide negative pressure wound therapy by reducing the pressure at wound 114. Therapy device 102 can draw a vacuum at wound 114 (relative to atmospheric pressure) by removing wound exudate, air, and other fluids from wound 114. Wound exudate may include fluid that filters from a patient's circulatory system into lesions or areas of inflammation. For example, wound exudate may include water and dissolved solutes such as blood, plasma proteins, white blood cells, platelets, and red blood cells. Other fluids removed from wound 114 may include instillation fluid 105 previously delivered to wound 114. Instillation fluid 105 can include, for example, a cleansing fluid, a prescribed fluid, a medicated fluid, an antibiotic fluid, or any other type of fluid which can be delivered to wound 114 during wound treatment. Instillation fluid 105 may be held in an instillation fluid canister 104 and controllably dispensed to wound 114 via instillation fluid tubing 108. In some embodiments, instillation fluid canister 104 is detachable from therapy device 102 to allow canister 106 to be refilled and replaced as needed.

The fluids 107 removed from wound 114 pass through removed fluid tubing 110 and are collected in removed fluid canister 106. Removed fluid canister 106 may be a component of therapy device 102 configured to collect wound exudate and other fluids 107 removed from wound 114. In some embodiments, removed fluid canister 106 is detachable from therapy device 102 to allow canister 106 to be emptied and replaced as needed. A lower portion of canister 106 may be filled with wound exudate and other fluids 107 removed from wound 114, whereas an upper portion of canister 106 may be filled with air. Therapy device 102 can be configured to draw a vacuum within canister 106 by pumping air out of canister 106. The reduced pressure within canister 106 can be translated to wound dressing 112 and wound 114 via tubing 110 such that wound dressing 112 and wound 114 are maintained at the same pressure as canister 106.

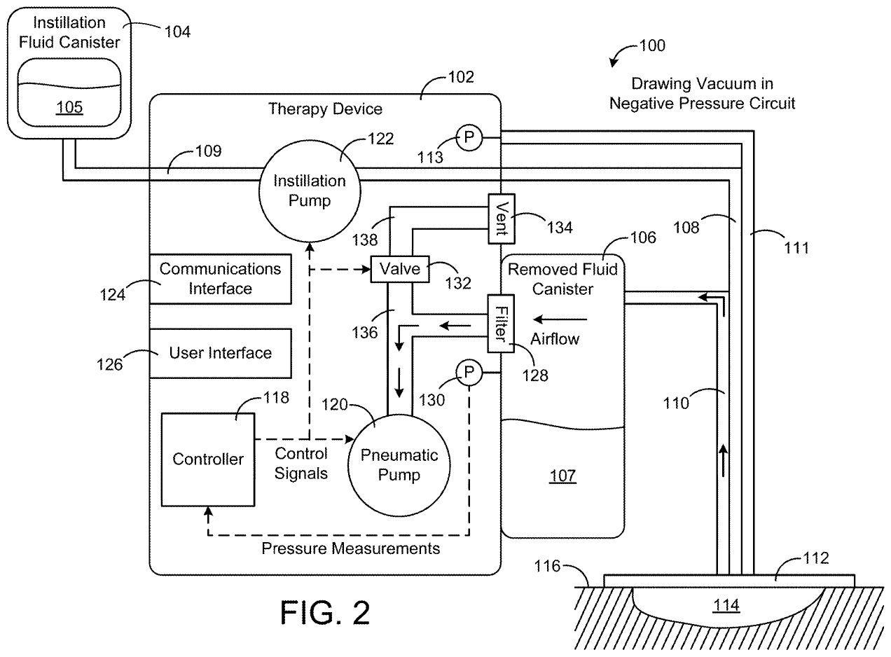

Referring particularly to FIGS. 2-4, block diagrams illustrating therapy device 102 in greater detail are shown, according to an exemplary embodiment. Therapy device 102 is shown to include a pneumatic pump 120, an instillation pump 122, a valve 132, a filter 128, and a controller 118. Pneumatic pump 120 can be fluidly coupled to removed fluid canister 106 (e.g., via conduit 136) and can be configured to draw a vacuum within canister 106 by pumping air out of canister 106. In some embodiments, pneumatic pump 120 is configured to operate in both a forward direction and a reverse direction. For example, pneumatic pump 120 can operate in the forward direction to pump air out of canister 106 and decrease the pressure within canister 106. Pneumatic pump 120 can operate in the reverse direction to pump air into canister 106 and increase the pressure within canister 106. Pneumatic pump 120 can be controlled by controller 118, described in greater detail below.

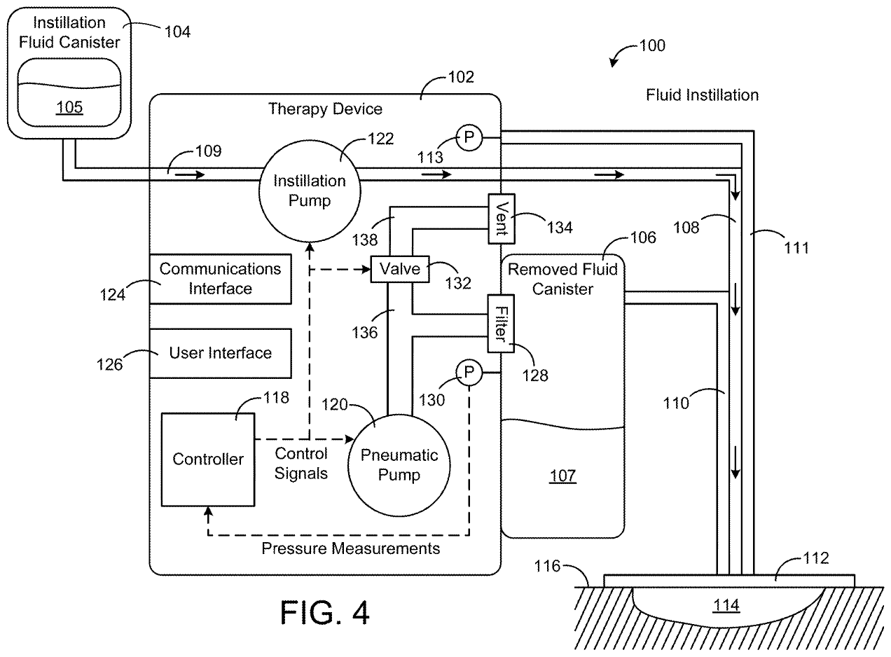

Similarly, instillation pump 122 can be fluidly coupled to instillation fluid canister 104 via tubing 109 and fluidly coupled to wound dressing 112 via tubing 108. Instillation pump 122 can be operated to deliver instillation fluid 105 to wound dressing 112 and wound 114 by pumping instillation fluid 105 through tubing 109 and tubing 108, as shown in FIG. 4. Instillation pump 122 can be controlled by controller 118, described in greater detail below.

Filter 128 can be positioned between removed fluid canister 106 and pneumatic pump 120 (e.g., along conduit 136) such that the air pumped out of canister 106 passes through filter 128. Filter 128 can be configured to prevent liquid or solid particles from entering conduit 136 and reaching pneumatic pump 120. Filter 128 may include, for example, a bacterial filter that is hydrophobic and/or lipophilic such that aqueous and/or oily liquids will bead on the surface of filter 128. Pneumatic pump 120 can be configured to provide sufficient airflow through filter 128 that the pressure drop across filter 128 is not substantial (e.g., such that the pressure drop will not substantially interfere with the application of negative pressure to wound 114 from therapy device 102).

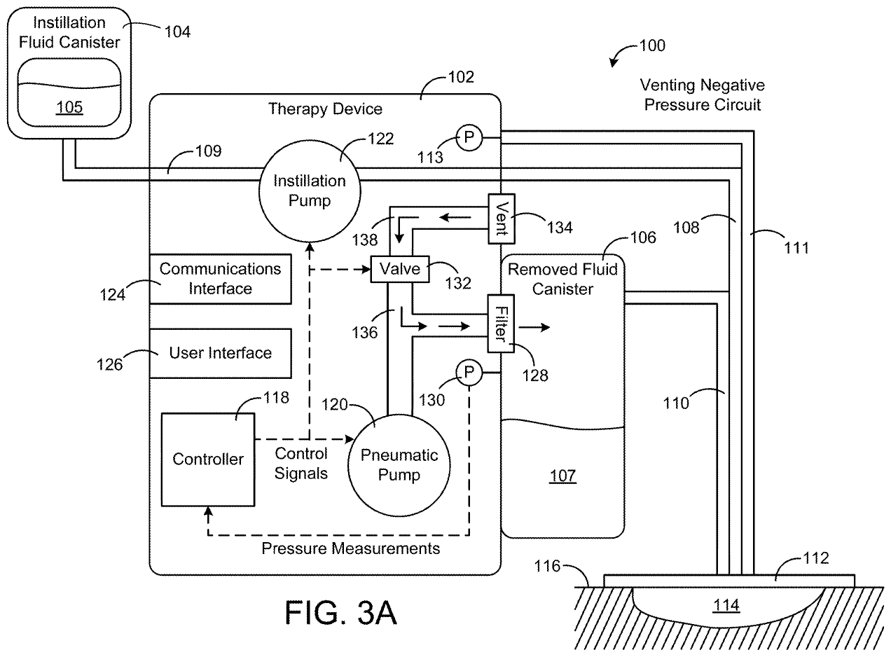

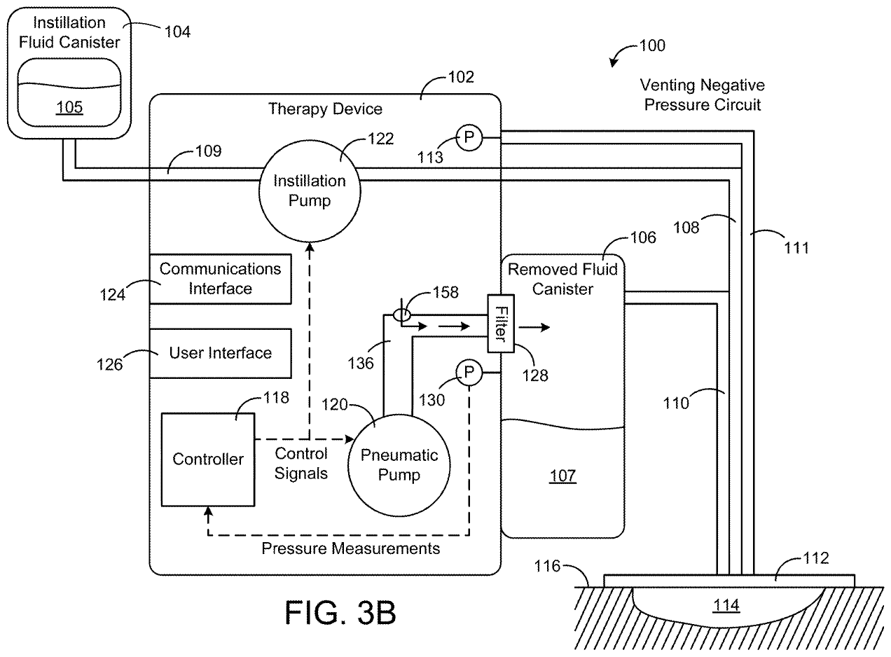

In some embodiments, therapy device 102 operates a valve 132 to controllably vent the negative pressure circuit, as shown in FIG. 3A. Valve 132 can be fluidly connected with pneumatic pump 120 and filter 128 via conduit 136. In some embodiments, valve 132 is configured to control airflow between conduit 136 and the environment around therapy device 102. For example, valve 132 can be opened to allow airflow into conduit 136 via vent 134 and conduit 138, and closed to prevent airflow into conduit 136 via vent 134 and conduit 138. Valve 132 can be opened and closed by controller 118, described in greater detail below. When valve 132 is closed, pneumatic pump 120 can draw a vacuum within a negative pressure circuit by causing airflow through filter 128 in a first direction, as shown in FIG. 2. The negative pressure circuit may include any component of system 100 that can be maintained at a negative pressure when performing negative pressure wound therapy (e.g., conduit 136, removed fluid canister 106, tubing 110, wound dressing 112, and/or wound 114). For example, the negative pressure circuit may include conduit 136, removed fluid canister 106, tubing 110, wound dressing 112, and/or wound 114. When valve 132 is open, airflow from the environment around therapy device 102 may enter conduit 136 via vent 134 and conduit 138 and fill the vacuum within the negative pressure circuit. The airflow from conduit 136 into canister 106 and other volumes within the negative pressure circuit may pass through filter 128 in a second direction, opposite the first direction, as shown in FIG. 3A.

In some embodiments, therapy device 102 vents the negative pressure circuit via an orifice 158, as shown in FIG. 3B. Orifice 158 may be a small opening in conduit 136 or any other component of the negative pressure circuit (e.g., removed fluid canister 106, tubing 110, tubing 111, wound dressing 112, etc.) and may allow air to leak into the negative pressure circuit at a known rate. In some embodiments, therapy device 102 vents the negative pressure circuit via orifice 158 rather than operating valve 132. Valve 132 can be omitted from therapy device 102 for any embodiment in which orifice 158 is included. The rate at which air leaks into the negative pressure circuit via orifice 158 may be substantially constant or may vary as a function of the negative pressure, depending on the geometry of orifice 158. For embodiments in which the leak rate via orifice 158 is variable, controller 118 can use a stored relationship between negative pressure and leak rate to calculate the leak rate via orifice 158 based measurements of the negative pressure. Regardless of whether the leak rate via orifice 158 is substantially constant or variable, the leakage of air into the negative pressure circuit via orifice 158 can be used to generate a pressure decay curve for use in estimating volume 160 (see FIG. 8) of wound 114.

In some embodiments, therapy device 102 includes a variety of sensors. For example, therapy device 102 is shown to include a pressure sensor 130 configured to measure the pressure within canister 106 and/or the pressure at wound dressing 112 or wound 114. In some embodiments, therapy device 102 includes a pressure sensor 113 configured to measure the pressure within tubing 111. Tubing 111 may be connected to wound dressing 112 and may be dedicated to measuring the pressure at wound dressing 112 or wound 114 without having a secondary function such as channeling installation fluid 105 or wound exudate. In various embodiments, tubing 108, 110, and 111 may be physically separate tubes or separate lumens within a single tube that connects therapy device 102 to wound dressing 112. Accordingly, tubing 110 may be described as a negative pressure lumen that functions apply negative pressure wound dressing 112 or wound 114, whereas tubing 111 may be described as a sensing lumen configured to sense the pressure at wound dressing 112 or wound 114. Pressure sensors 130 and 113 can be located within therapy device 102, positioned at any location along tubing 108, 110, and 111, or located at wound dressing 112 in various embodiments. Pressure measurements recorded by pressure sensors 130 and/or 113 can be communicated to controller 118. Controller 118 use the pressure measurements as inputs to various pressure testing operations and control operations performed by controller 118.

Controller 118 can be configured to operate pneumatic pump 120, instillation pump 122, valve 132, and/or other controllable components of therapy device 102. In some embodiments, controller 118 performs a pressure testing procedure by applying a pressure stimulus to the negative pressure circuit. For example, controller 118 may instruct valve 132 to close and operate pneumatic pump 120 to establish negative pressure within the negative pressure circuit. Once the negative pressure has been established, controller 118 may deactivate pneumatic pump 120. Controller 118 may cause valve 132 to open for a predetermined amount of time and then close after the predetermined amount of time has elapsed.

In some embodiments, therapy device 102 includes a user interface 126. User interface 126 may include one or more buttons, dials, sliders, keys, or other input devices configured to receive input from a user. User interface 126 may also include one or more display devices (e.g., LEDs, LCD displays, etc.), speakers, tactile feedback devices, or other output devices configured to provide information to a user. In some embodiments, the pressure measurements recorded by pressure sensors 130 and/or 113 are presented to a user via user interface 126. User interface 126 can also display alerts generated by controller 118. For example, controller 118 can generate a “no canister” alert if canister 106 is not detected.

In some embodiments, therapy device 102 includes a data communications interface 124 (e.g., a USB port, a wireless transceiver, etc.) configured to receive and transmit data. Communications interface 124 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, communications interface 124 can include a USB port or an Ethernet card and port for sending and receiving data via an Ethernet-based communications link or network. In another example, communications interface 124 can include a Wi-Fi transceiver for communicating via a wireless communications network or cellular or mobile phone communications transceivers.

NPWT Device with Noise Control

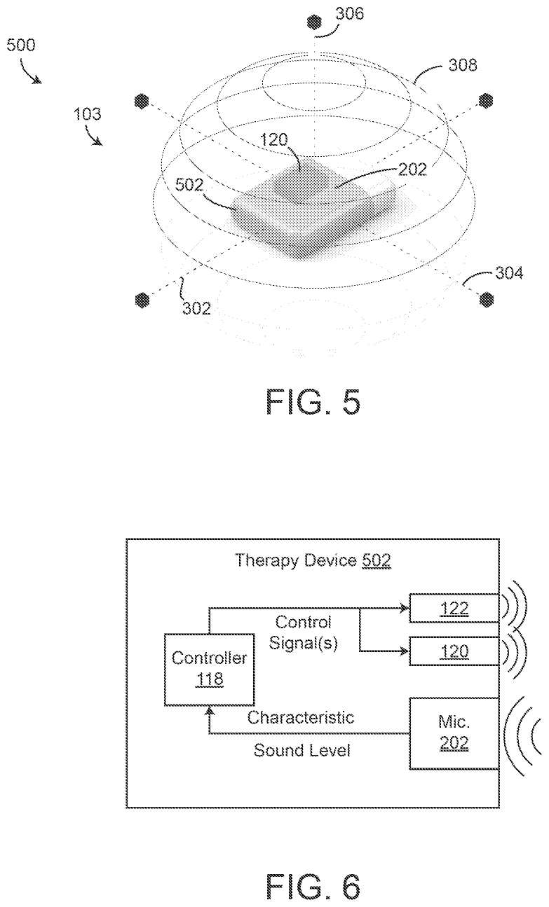

Referring to FIGS. 5-21, systems and methods for a NPWT device with controlled noise (e.g., noise cancellation, pump adjustment, etc.) are shown. The NPWT device as described herein with reference to FIGS. 5-21 can be the same as or similar to the therapy device 102 as described in greater detail above with reference to FIGS. 1-4, and may include any of the components of the therapy device 102. Similarly, the NPWT device as described herein with reference to FIGS. 5-21 can be configured for use with the dressing 112 as described in greater detail above.

NPWT Device with Active Pump Control

Referring to FIGS. 5-9, an embodiment of the therapy device 102, shown as therapy device 502, is configured to provide noise control in addition to negative pressure wound therapy. In some embodiments, the therapy device 502 is the same as or similar to the therapy device 102. In some embodiments, the therapy device 502 includes the pneumatic pump 120 for drawing a negative pressure at a dressing (e.g., the dressing 112). The therapy device 502 also includes a single microphone 202 that is configured to monitor a sound level, a decibel level, a sound amplitude, etc., of sound in an environment 103 of the therapy device 102, according to some embodiments. In some embodiments, the microphone 202 is also configured to measure a characteristic of sound in the environment 103 of the therapy device 502. For example, the microphone 202 is configured to measure a frequency, phase, amplitude, etc., or other characteristic of sound in the environment 103 of the therapy device 502.

When the pneumatic pump 120 operates to draw a negative pressure at a dressing, the pneumatic pump 120 may emit sound 308 into the environment 103 of the therapy device 502. It may be desirable to reduce, minimize, mitigate, or otherwise control the sound emitted by the pneumatic pump 120. For example, in a public environment or during night time, a user's experience may be improved by mitigating noise emitted by the pneumatic pump 120. The noise or sound emitted by the pneumatic pump 120 as it operates can be controlled or reduced to an acceptable level by adjusting operation of the pneumatic pump 120, providing predetermined or active noise cancellation, or a combination thereof, according to some embodiments.

Referring still to FIG. 5, the therapy device 502 may define a first axis 302, a second axis 304, and a third axis 306, according to some embodiments. In some embodiments, the sound emitted by the pneumatic pump 120 is along or in a direction along the third axis 306. The microphone 202 may be a directional microphone that is configured to measure the sound level and/or the characteristic of the sound in the environment in a particular direction (e.g., along the third axis 306), or may be a non-directional microphone that measures overall sound level (e.g., ambient sound level) of the environment 103.

As shown in FIG. 6, the therapy device 502 includes at least the controller 118, the microphone 202, the pneumatic pump 120, and the instillation pump 122, according to some embodiments. In some embodiments, the controller 118 is configured to obtain the sound level and/or the characteristic of the sound in the environment 103 from the microphone 202. In some embodiments, the controller 118 is configured to use the sound level and/or the characteristic of the sound in the environment 103 to determine or generate control signals for the instillation pump 122 and/or the pneumatic pump 120 to mitigate or reduce sound output by the pumps 120 and 122. In some embodiments, the therapy device 502 is configured to adjust a duty cycle of the pump 120 and/or a target pressure of the pneumatic pump 120 based on the sound level measured by the microphone 202 (e.g., in response to a determination that the sound level provided by the microphone 202 exceeds a threshold).

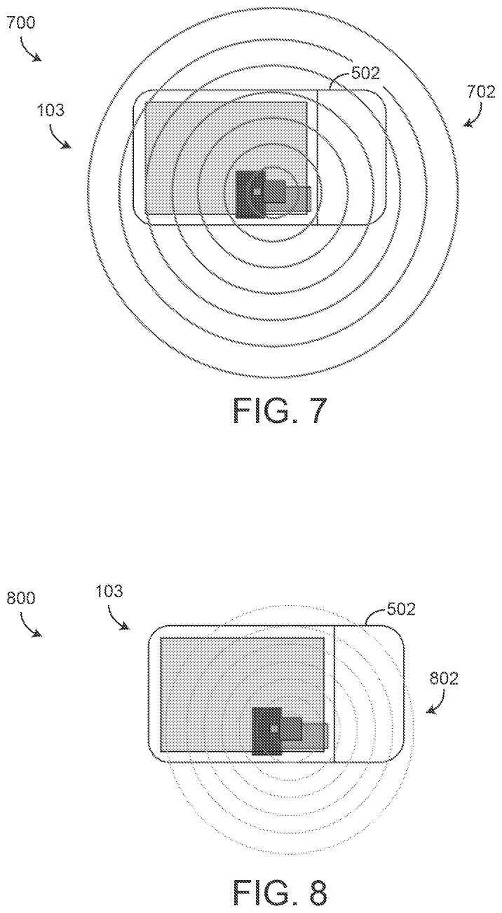

Referring to FIG. 7, a diagram 700 shows the pneumatic pump 120 emitting sound waves 702 into the environment 103 as the pneumatic pump 120 operates according to a first duty cycle to draw a negative pressure at a wound dressing. If the sound waves 702 emitted into the environment are determined by the controller 118, based on the measurement of the microphone 202, to exceed a threshold, the duty cycle of the pneumatic pump 120 can be adjusted to reduce the sound level of the sound waves 702.

Referring to FIG. 8, a diagram 800 shows the pneumatic pump 120 emitting sound waves 802 into the environment 103 as the pneumatic pump 120 operates according to a second duty cycle to draw a negative pressure at a wound dressing. In some embodiments, the controller 118 is configured to adjust operation of the pneumatic pump 120 from the first duty cycle as shown in FIG. 7 to the second duty cycle as shown in FIG. 8 to reduce the sound level in the environment 103. As illustrated by FIG. 8, the sound waves 802 have a reduced sound level which can be measured by the microphone 202, according to some embodiments. In some embodiments, if the sound waves 802 have a sound level that is less than the threshold, the controller 118 continues operating the pneumatic pump 120 according to the second duty cycle as shown in FIG. 8. In some embodiments, if the sound waves 802 have a sound level that is significantly less than the threshold, the controller 118 may adjust the duty cycle to a third duty cycle (e.g., an increase duty cycle that is greater than the second duty cycle but less than the first duty cycle). In some embodiments, the second duty cycle as illustrated in FIG. 8 is less than the first duty cycle as illustrated in FIG. 7. If the sound level of the environment 103 as measured by the microphone 202 still exceeds the threshold, the controller 118 may reduce a target pressure, drawdown pressure, or a magnitude of the negative pressure provided by the pneumatic pump 120.

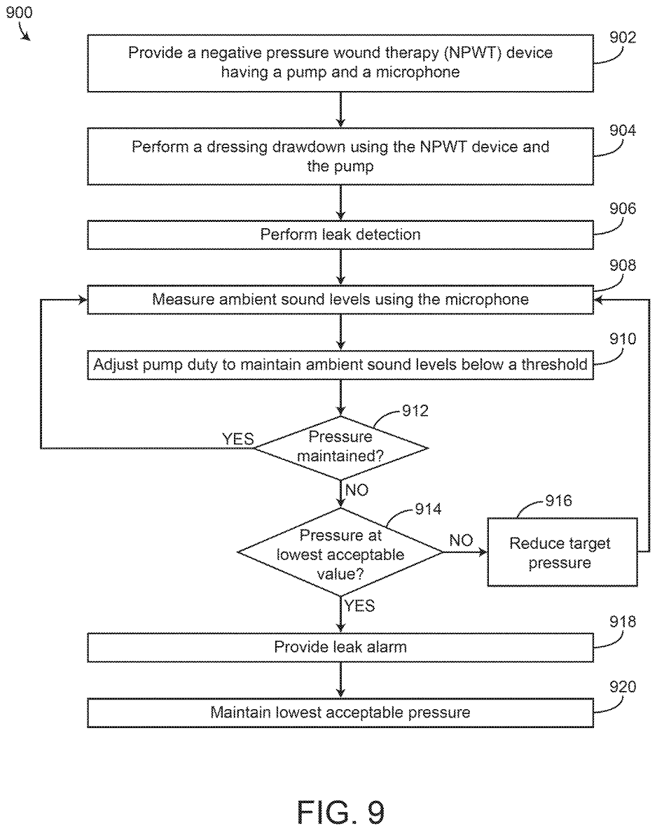

Referring to FIG. 9, a process 900 for controlling or operating the therapy device 502 as shown in FIGS. 5-8 includes steps 902-920, according to some embodiments. In some embodiments, process 900 can be performed by the therapy device 502 or the controller 118 as shown in FIGS. 5-8. The process 900 can be performed to adjust operation of the therapy device 502 or the pneumatic pump 120 thereof based on currently measured environmental sound level or properties to mitigate or reduce the sound produced by operation of the pneumatic pump 120.

Process 900 includes providing a negative pressure wound therapy (NPWT) device having a pump and a microphone (step 902), according to some embodiments. Step 902 can include providing the therapy device 502 and the dressing 112, with the therapy device 502 operably coupled with the dressing 112 so that the pneumatic pump 120 of the therapy device 502 is configured to draw a negative pressure at the dressing 112. The microphone can be configured to measure ambient environmental sound levels surrounding the therapy device, according to some embodiments. The measurements of the microphone can be used by a controller, processing circuit, or processing circuitry of the NPWT device for use in adjusting operation of the pneumatic pump.

Process 900 includes performing a dressing drawdown using the NPWT device and the pump (e.g., the pneumatic pump 120) (step 904), according to some embodiments. In some embodiments, step 904 is performed by the pneumatic pump 120 at the wound dressing 112. The wound dressing 112 may seal with periwound tissue so that the pump is operable to draw a negative pressure at the wound. In some embodiments, step 904 includes continuously increasing operation of the pneumatic pump 120 until a pressure is achieved at the dressing 112 (e.g., using detected pressure as feedback).

Process 900 includes performing a leak detection (step 906), according to some embodiments. In some embodiments, the leak detection is a process that is performed by the controller 118 (e.g., based on sensor data obtained from a pressure sensor, based on feedback from the pneumatic pump 120, etc.). In some embodiments, step 906 includes the controller 118 monitoring pressure received from a pressure sensor as feedback, and if a certain pressure amount cannot be achieved, the controller 118 may determine that there is a leak at the dressing. In some embodiments, step 906 includes the controller 118 monitoring a duty cycle of the pneumatic pump 120 and determining, based on the duty cycle (e.g., if the duty cycle is too high) that there is a leak at the dressing 112.

Process 900 includes measuring ambient sound levels using the microphone (step 908), according to some embodiments. In some embodiments, step 908 includes obtaining a decibel level of an environment surrounding the NPWT device as the pneumatic pump 120 operates to draw the negative pressure at the dressing. In some embodiments, step 908 includes obtaining time-series values of the sound level in real-time by the microphone 202 as the NPWT device operates.

Process 900 includes adjusting a pump duty to maintain ambient sound levels below a threshold (step 910), according to some embodiments. In some embodiments, step 910 includes comparing the ambient sound level as measured by the microphone in step 908 to a threshold amount. If the ambient sound level approaches or exceeds the threshold, the controller 118 may decrease the duty cycle to reduce the sound level and maintain the sound level below the threshold. In some embodiments, steps 908 and 910 are performed concurrently or in real-time. Step 910 can include generating a duty cycle signal and providing the duty cycle signal to the pump. In some embodiments, step 910 is performed by the controller 118.

Process 900 includes determining if the pressure is maintained at the dressing (step 912), according to some embodiments. In some embodiments, step 912 is performed by the controller 118. Step 912 can include monitoring the pressure of the dressing over time and determining if the pressure is maintained. If the pressure is not maintained (step 912, “NO”), process 900 proceeds to step 914. If the pressure is maintained (step 912, “YES”), process 900 returns to step 908. In some embodiments, step 912 includes determining if a target pressure is achieved and/or maintained over time at the dressing (e.g., is the pressure is maintained at the target pressure for a predetermined amount of time).

Process 900 includes determining if the pressure is at a lowest acceptable value (step 914), according to some embodiments. In some embodiments, step 914 is performed by the controller 118 and is performed if a current target pressure cannot be maintained (step 912, “NO”). In some embodiments, step 914 includes comparing a current pressure or a current target pressure of the dressing 112 to a lowest acceptable value. If the current pressure or the current target pressure is not at the lowest acceptable value (e.g., a lowest magnitude of negative pressure) (step 914 “NO”), process 900 proceeds to step 916. If the current pressure or the current target pressure is at the lowest acceptable value (e.g., a lowest magnitude of negative pressure) (step 914 “YES”), process 900 proceeds to step 918.

Process 900 includes reducing a target pressure (step 916) in response to the pressure not being at the lowest acceptable pressure (step 914, “NO”), according to some embodiments. In some embodiments, step 916 includes reducing the target pressure by a predetermined or an adjustment amount. In some embodiments, in response to performing the step 916, process 900 returns to step 908.

Process 900 includes providing a leak alarm (step 918) in response to the pressure being at the lowest acceptable value (step 914, “YES”) and the pressure not being maintained at the dressing (step 912, “NO”), according to some embodiments. In some embodiments, step 918 is performed by the controller 118 and the user interface 126 of the NPWT device 102 or the therapy device 502. In some embodiments, providing the leak alarm includes providing a notification, an alert, a display, etc., that is visual and/or aural via the user interface 1256. In some embodiments, the leak alarm is provided by transmitting a signal (e.g., wirelessly) to a user device such as a smartphone, a central operating station, a tablet, a medical system, etc.

Process 900 includes maintaining the lowest acceptable pressure (step 920), according to some embodiments. In some embodiments, step 920 is performed by the NPWT device or the pneumatic pump thereof. In some embodiments, step 920 is performed in response to the pressure or the target pressure being at a lowest acceptable value and the pressure not being maintained. In some embodiments, step 920 is performed to provide a minimal amount of negative pressure in the case of a leak condition.

NPWT Device with Active Pump Control and Speaker

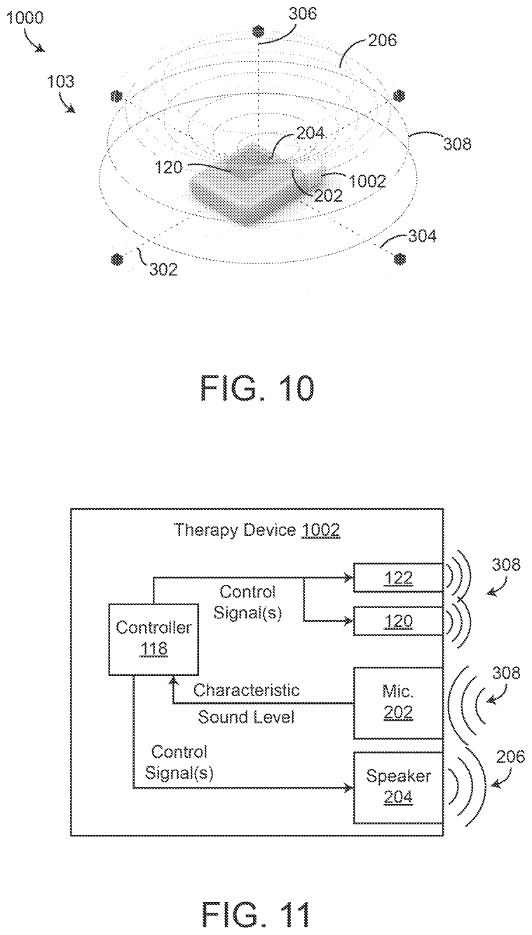

Referring to FIGS. 10-13, an embodiment of the therapy device 102, shown as therapy device 1002, is configured to provide noise control in addition to negative pressure wound therapy. In some embodiments, the therapy device 1002 is the same as or similar to the therapy device 102 and may include any of the same or similar structure as the therapy device 102 with additional components for noise control (e.g., for noise cancellation). In some embodiments, the therapy device 1002 includes the pneumatic pump 120 for drawing a negative pressure at a dressing (e.g., the dressing 112). The therapy device 1002 also includes the single microphone 202 and a single speaker 204, according to some embodiments. In some embodiments, the single speaker 204 is configured to emit sound or sound waves into the environment 103 of the therapy device 1002 to provide noise cancellation. In some embodiments, the microphone 202 is configured to monitor a sound level and a characteristic of sound in the environment 103 (e.g., a phase, a frequency, etc.). In some embodiments, the speaker 204 is configured to be operated according to a selected, predefined, or predetermined profile to provide sound waves into the environment 103. The sounds or sound waves provided by the speaker 204 are configured to interfere with the sound waves produced by operation of the pneumatic pump 120 of the therapy device 1002 to provide noise cancellation to reduce sounds emitted into the environment 103 by the pneumatic pump 120, according to some embodiments.

Referring particularly to FIG. 10, the pneumatic pump 120 is shown generally emitting sound waves 308 into the environment 103, according to some embodiments. In some embodiments, the pneumatic pump 120 emits the sound waves 308 generally along all of the axes 302-306. In some embodiments, the speaker 204 is configured to emit sound waves 206 along the third axis 306 to provide noise cancellation of the sound waves 308. In some embodiments, the sound waves 206 are configured to interfere with the sound waves 308 to provide noise cancellation. In some embodiments, the speaker 204 operates according to a predetermined or a predefined profile to emit the sound waves 206 for noise cancellation (e.g., to provide sound waves having a phase offset that destructively interfere with the sound waves 308 emitted by the pneumatic pump 120).

Referring particularly to FIG. 11, a block diagram of the therapy device 1002 is shown, according to some embodiments. The therapy device 1002 includes the controller 118, the pumps 120-122, the microphone 202, and the speaker 204, according to some embodiments. In some embodiments, the controller 118 is configured to use a current operational status of the pumps 120 and 122, and the sound level or characteristic as provided by the microphone 202 to select from different predetermined or predefined operational profiles for the speaker 204. In some embodiments, the predetermined or predefined operational profiles are stored in memory of the controller 118, and processing circuitry of the controller 118 selects from the predetermined or predefined operational profiles for the speaker 204. In some embodiments, the controller 118 operates the speaker 204 according to the selected predetermined or predefined operational profile so that the speaker 204 emits the sound waves 206 into the environment 103 to provide noise cancellation.

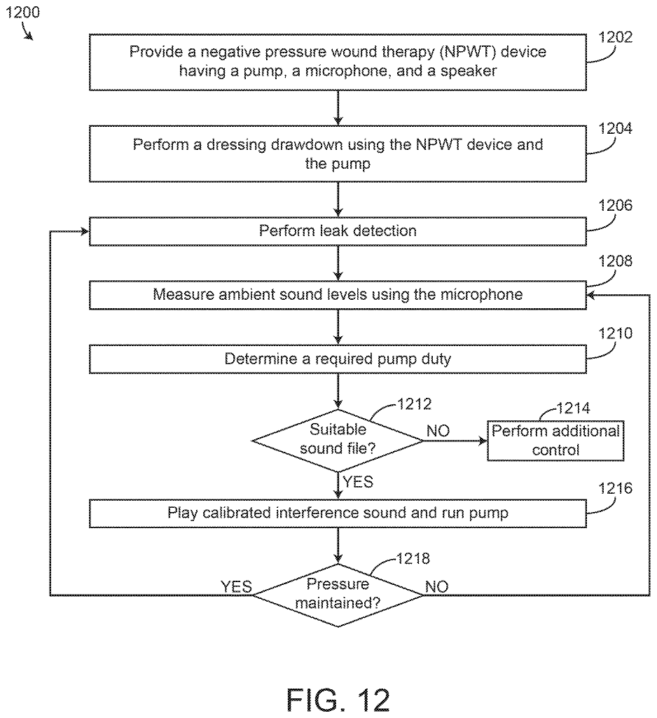

Referring particularly to FIG. 12, a process 1200 for operating or using the therapy device 1002 is shown, according to some embodiments. In some embodiments, the process 1200 includes steps 1202-1218 and at least some of the steps 1202-1218 are performed by the therapy device 1002 as described in greater detail above with reference to FIGS. 10-11. In some embodiments, the process 1200 is performed to facilitate noise reduction using a single speaker and a single microphone (e.g., the speaker 204 and the microphone 202).

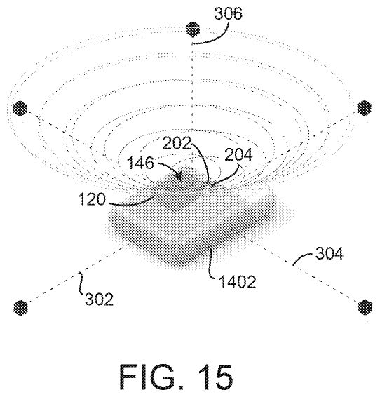

Process 1200 includes providing a negative pressure wound therapy (NPWT) device having a pump, a microphone, and a speaker (step 1202), according to some embodiments. In some embodiments, the NPWT device of process 1200 is the therapy device 1002 as described in greater detail above with reference to FIGS. 10-11 (or the therapy device 1502 as shown in FIG. 15 below). In some embodiments, the NPWT device is configured to fluidly couple with a dressing (e.g., the wound dressing 112) to draw a negative pressure at the dressing to provide NPWT to a patient's wound. In some embodiments, the NPWT device is provided as a kit.

Process 1200 includes performing a dressing drawdown using the NPWT device and the pump (e.g., the pneumatic pump 120) (step 1204), according to some embodiments. In some embodiments, step 1204 is the same as or similar to step 904 of process 900.

Process 1200 includes performing a leak detection (step 1206), according to some embodiments. In some embodiments, step 1206 is the same as or similar to step 906 of process 900 as described in greater detail above with reference to FIG. 9. In some embodiments, step 1206 includes monitoring a pressure of a dressing at which the therapy device operates to draw the negative pressure and determining if the pressure is maintained at the dressing.

Process 1200 includes measuring ambient sound levels using the microphone (step 1208), according to some embodiments. In some embodiments, step 1208 is the same as or similar to step 908 of process 900. In some embodiments, step 1208 includes using the microphone (e.g., the microphone 202) to record or obtain the sound level of the environment and/or a characteristic (e.g., a phase, an amplitude, a frequency, etc.) of sound in the environment of the NPWT device.

Process 1200 includes determining a required pump duty (step 1210), according to some embodiments. In some embodiments, step 1210 includes determining or adjusting a pump duty for the pump (e.g., the pneumatic pump 120) to maintain or achieve a desired target pressure (e.g., a target negative pressure) at the dressing (e.g., the wound dressing 112). In some embodiments, step 1210 includes determining a pump duty cycle to reduce sound output and to achieve the desired target pressure. In some embodiments, step 1210 is the same as or similar to step 910 of process 900. In some embodiments, step 1210 is performed by the controller 118.

Process 1200 includes determining if a suitable sound file (e.g., a predetermined profile) is available (step 1212), according to some embodiments. In some embodiments, step 1212 includes using the pump duty as determined in step 1210 to select a corresponding sound profile for the speaker 204. In some embodiments, step 1212 includes selecting the sound profile based on the determined pump duty so that the speaker 204 will be operated to provide sound waves for noise cancellation into the environment surrounding the NPWT device.

Process 1200 includes playing a calibrated interference sound and running the pump (step 1216) in response to finding a suitable sound file (step 1212, “YES”), according to some embodiments. In some embodiments, step 1218 is performed by the controller 118 and the speaker 204 using the sound profile selected in step 1212. In some embodiments, the sound profile is selected for a particularly expected frequency, phase, and/or amplitude of sound that is expected to be emitted by the pneumatic pump 120 for the determined pump duty. In

Process 1200 includes performing additional control (step 1214) in response to not finding a suitable sound file (step 1212, “NO”), according to some embodiments. In some embodiments, performing additional control (e.g., performing step 1214) includes adjusting operation of the pump (e.g., adjusting a pump duty) and returning to step 1208. In some embodiments, step 1214 is performed by the controller 118.

Process 1200 includes playing a calibrated interference sound and running the pump (e.g., to draw negative pressure) (step 1216) in response to a suitable sound file being found (step 1212, “YES”), according to some embodiments. In some embodiments, step 1216 includes operating the speaker 204 to produce, output, or otherwise emit sound according to the suitable sound file so that the sound emitted by the speaker 204 cancels or interferes destructively with sound emitted by the pump as the pump operates to draw negative pressure. In some embodiments, step 1216 is performed by the speaker 204 and the pneumatic pump 120.

Process 1200 includes determining if pressure is maintained at the dressing (step 1218), according to some embodiments. In some embodiments, step 1218 is performed based on monitoring pressure readings provided by pressure sensors 130 and/or 113. In some embodiments, if the pressure readings indicate that the pressure is stable over time (e.g., does not deviate more than a predetermined amount), this indicates that the pressure is maintained at the dressing (step 1218, “YES”) and process 1200 returns to step 1206. In some embodiments, if the pressure readings indicate that the pressure is not stable over time (e.g., deviates more than a predetermined amount), this indicates that the pressure is not maintained at the dressing (step 1218, “NO”), and process 1200 returns to step 1208.

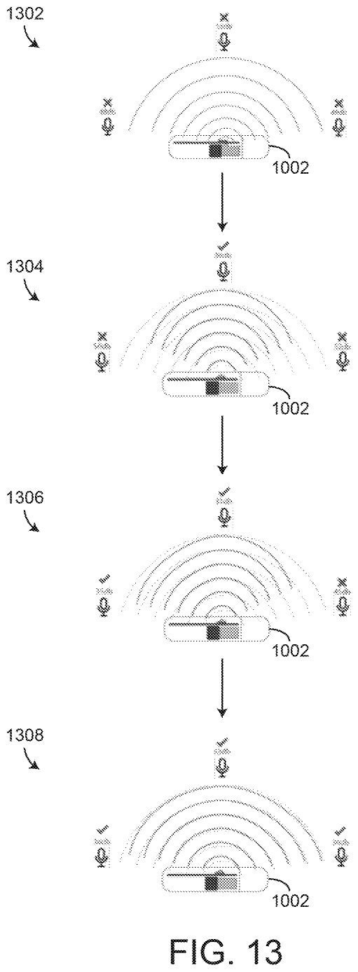

FIG. 13 illustrates different parts of the process 1200, according to some embodiments. For example, diagram 1302 illustrates the therapy device 1002 operating to draw a negative pressure at a dressing and emitting sound into environment surrounding the therapy device 1002. Diagram 1304 illustrates the therapy device 1002 operating to draw the negative pressure and operating the speaker 204 to emit sound according to a first selected profile which does not achieve optimal noise cancellation. Diagram 1306 illustrates the speaker 204 of the therapy device 1002 being operated to emit sound according to a second selected profile which also does not achieve optimal noise cancellation. Diagram 1308 illustrates the speaker 204 of the therapy device 1002 being operated to emit sound according to a third selected profile which achieves optimal or best possible noise cancellation.

NPWT Device with Sound Absorbing Chamber

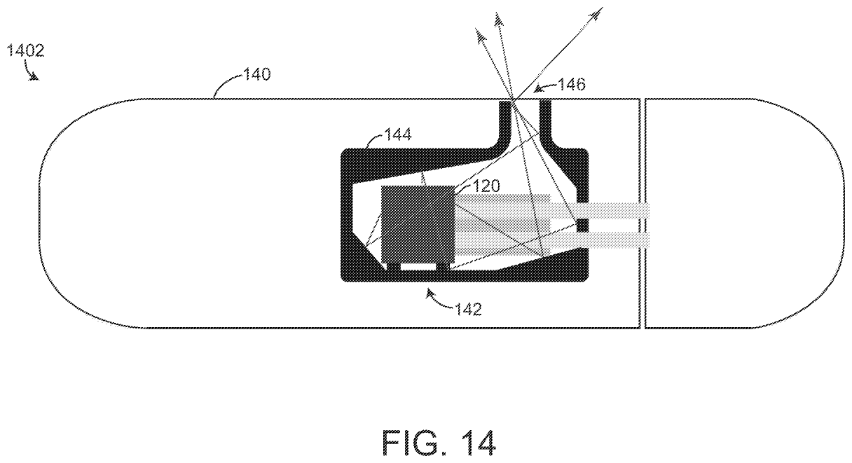

Referring to FIG. 14, a therapy device 1402 is shown according to another embodiment. The therapy device 1402 can be the same as or similar to the therapy device 1002 or the therapy device 102 as described in greater detail above with reference to FIGS. 1-13, and may include any of the components of the therapy device 102 or the therapy device 1002 as described in greater detail above. In the embodiment shown in FIG. 14, the pneumatic pump 120 (and/or the instillation pump 122) is shown positioned within a sound absorbing chamber 142 that is configured to absorb or deaden sound emitted by the pneumatic pump 120 as it operates to draw the negative pressure. In some embodiments, the sound absorbing chamber 142 includes an inner volume within which the pneumatic pump 120 is positioned. The sound absorbing chamber 142 includes sound absorbing materials 144 disposed about sidewalls of the sound absorbing chamber 142, according to some embodiments. In some embodiments, the sound absorbing materials 144 are configured to absorb sound or vibrations of the pneumatic pump 120 as the pneumatic pump 120 operates to draw the negative pressure or otherwise operates. In some embodiments, the sound absorbing materials 144 include foam, felt, a dense material, etc., or any other material configured to absorb sound or vibrations. In some embodiments, the sound absorbing chamber 142 has geometry so that sound emitted by the pneumatic pump 120 is reflected inwards towards another one of inner walls of the sound absorbing chamber 142. In some embodiments, the sound absorbing chamber 142 has an opening 146 through which sound is directed into the environment or surroundings of the therapy device 1402.

In some embodiments, the opening 146 is positioned on a face of the therapy device 1402 so that sound is emitted in a generally outwards direction from the therapy device 1402 into the environment of the therapy device 1402. Advantageously, using the sound absorbing chamber 142 can reduce sound levels of sound produced by the pneumatic pump 120 before the sound is emitted into the environment surrounding the therapy device 1402. In some embodiments, the opening 146 also facilitates a known location at which the sound of the pneumatic pump 120 will originate in the environment surrounding the therapy device 1402. In some embodiments, the speaker 204 can be positioned adjacent or proximate the opening 146 to facilitate improved noise cancellation. Similarly, a microphone for monitoring sound level and/or characteristics of the sound may be positioned proximate the opening 146.

Referring to FIG. 15, the therapy device 1402 is shown including the pneumatic pump 120 positioned within the sound absorbing chamber 142, the speaker 204 positioned proximate the opening 146, and the microphone 202 positioned proximate the opening 146. In some embodiments, the opening 146 is positioned on a top of the therapy device 1402 so that the sound emitted by the pneumatic pump 120 is emitted in a generally vertical direction (e.g., along the third axis 306). In some embodiments, the microphone 202 and the speaker 204 are positioned proximate the opening 146 and oriented such that the speaker 204 emits sound for noise cancellation in the vertical direction along the third axis 306. In some embodiments, the therapy device 1402 as described herein that includes the sound absorbing chamber 142 is operated the same as or similarly to the therapy device 1002 as described in greater detail above with reference to FIGS. 10-13.

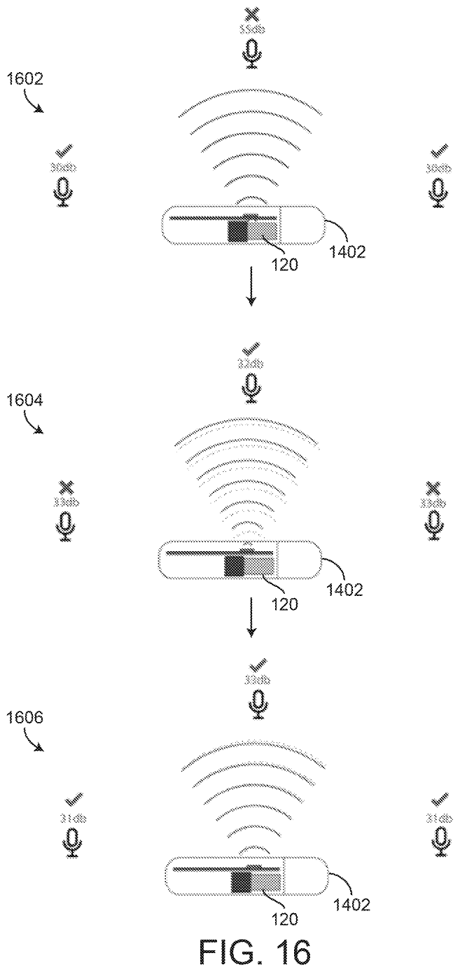

FIG. 16 provides various illustrations of a process for operating the therapy device 1402 to provide noise cancellation, according to some embodiments. For example, diagram 1602 shows the therapy device 1402 operating to draw negative pressure and emitting sound into the environment or surroundings of the therapy device 1402 in a generally vertical or upwards direction. Diagram 1604 illustrates the speaker 204 operating to provide sound waves into the environment to provide noise cancellation for the sound emitted by the pneumatic pump 120. In FIG. 16, the speaker 204 emits sound waves that are out of phase with the sound waves emitted by the pneumatic pump 120, and therefore require adjustment to achieve optimal or best possible noise cancellation. Diagram 1606 of FIG. 16 illustrates the speaker 204 operating to provide sound for noise cancellation that are at an appropriate phase for noise cancellation of the sound emitted by the pneumatic pump 120. As shown in diagram 1606, the speaker 204 emits sound waves in the same direction and generally at the same phase as the sound waves emitted by the pneumatic pump 120 to thereby provide noise cancellation.

NPWT Device with Active Pump Control and Multiple Speakers

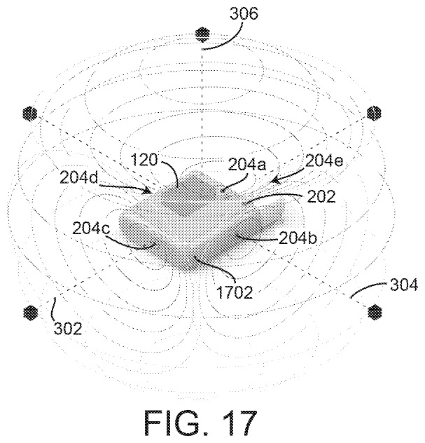

Referring to FIG. 17, a therapy device 1702 is shown including a single microphone 202, and multiple speakers 204, according to some embodiments. In some embodiments, the therapy device 1702 is the same as or similar to the therapy device 102, the therapy device 1002, or the therapy device 1402, and may include any of the structure, components, etc., of the therapy device 102, the therapy device 1002, or the therapy device 1402. The therapy device 1702 includes the pneumatic pump 120 that operates to draw a negative pressure and emits sound into the environment or surroundings of the therapy device 1702, according to some embodiments. The therapy device 1702 also includes the microphone 202 positioned to obtain sound level and/or information (e.g., a characteristic, phase, etc.) of sound in the environment. The therapy device 1702 further includes at least a first speaker 204a, a second speaker 204b, and a third speaker 204c. The therapy device 1702 may further include a fourth speaker 204d, and a fifth speaker 204e. In some embodiments, the first speaker 204a is positioned on a front face of the therapy device 1702 and is configured to emit sound in an upwards direction along the third axis 306 (e.g., in a same direction as the sound emitted by the pneumatic pump 120). The second speaker 204b can be positioned on a side of the therapy device 1702 and is configured to emit sound for noise cancellation along the second axis 304. The third speaker 204c is positioned on another side (e.g., a bottom) of the therapy device 1702 and is configured to emit sound for noise cancellation along the first axis 302 as shown. The fourth speaker 204d can be configured to emit sound for noise cancellation along the second axis 304 in an opposite direction as the second speaker 204b. The fifth speaker 204e can also be configured to emit sound for noise cancellation along the second axis 304 in an opposite direction as the third speaker 204c. In some embodiments, the first speaker 204a is a primary speaker for noise cancellation since noise cancellation is most efficient by emitting sound waves for destructive interference in a same direction as the sound waves emitted by the pneumatic pump 120. The second speaker 204b, the third speaker 204c, the fourth speaker 204d, and the fifth speaker 204c are configured to provide supplemental or additional noise cancellation in addition to the first speaker 204a.

In some embodiments, the speakers 204 are configured to be operated by the controller 118 similarly to the process 1200 as described in greater detail above with reference to FIG. 12. For example, the speakers 204 can be operated according to a selected profile or suitable sound file. In some embodiments, each of the speakers 204 are operated according to different profiles that are selected in unison (e.g., selecting a single profile that includes sub-profiles for each of the different speakers 204), or are individually or independently selected. In this way, the therapy device 1702 may be operated similarly to the therapy device 1002, according to some embodiments.

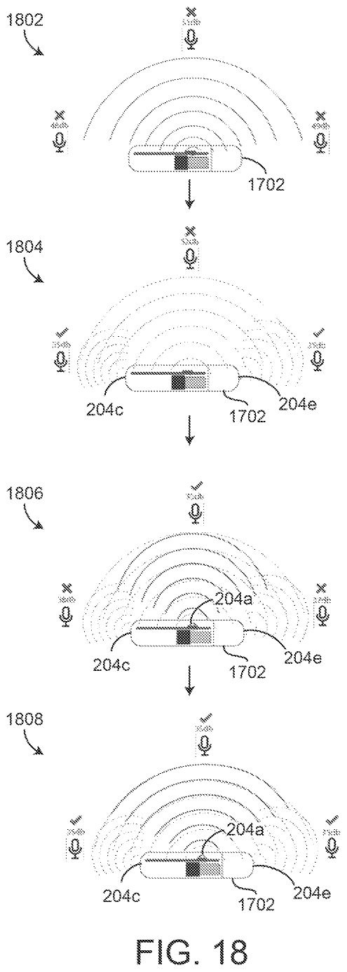

FIG. 18 shows different diagrams illustrating calibration and operation of the therapy device 1702, according to some embodiments. For example, diagram 1802 illustrates the therapy device 1702 emitting sound as the pneumatic pump 120 operates to draw negative pressure. Diagram 1804 illustrates one or more secondary speakers (e.g., speakers 204c and 204e) operating to provide supplemental noise cancellation. Diagram 1806 illustrates the primary speaker 204a and the one or more secondary speakers (e.g., speakers 204c and 204c) operating to provide noise cancellation for the sound waves emitted by the pneumatic pump 120. Diagram 1808 illustrates the primary speaker 204a and the secondary speakers (e.g., speakers 204c and 204c) operating to provide noise cancellation with the primary speaker 204a adjusted to provide sound waves for noise cancellation at an appropriate phase to cancel the sound emitted by the pneumatic pump 120.

NPWT Device with Active Pump Control, Multiple Microphones, and Multiple Speakers

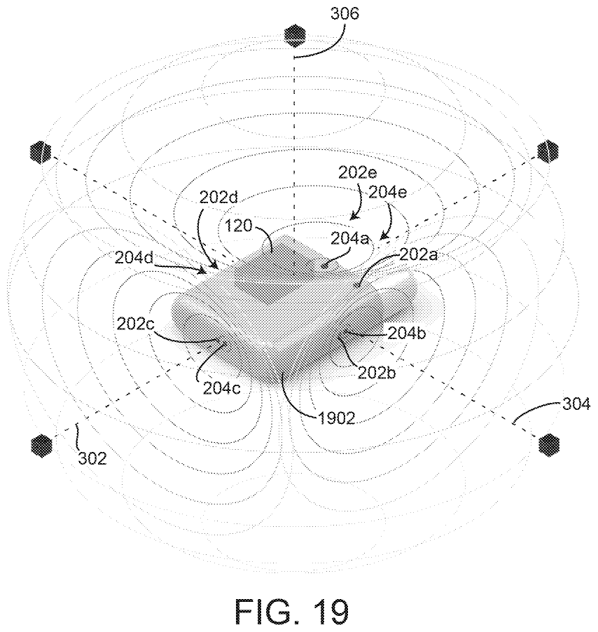

Referring to FIGS. 19-21, a therapy device 1902 is shown to include the pneumatic pump 120, multiple microphones 202, and multiple speakers 204, according to some embodiments. In some embodiments, each of the microphones 202 are positioned proximate a corresponding one of the speakers 204 and are configured to obtain sound level and/or a characteristic of sound in the environment that surrounds the therapy device 1902 proximate the speakers 204 to improve efficiency of noise cancellation performed by the speakers 204.

Referring particularly to FIG. 19, the therapy device 1902 includes the first speaker 204a, the second speaker 204b, the third speaker 204c, the fourth speaker 204d, and the fifth speaker 204c. It should be understood that the therapy device 1902 may have any number of speakers 204. For example, the therapy device 1902 may include only the first speaker 204a, the second speaker 204b, and the third speaker 204c. In some embodiments, the therapy device 1902 additionally includes the fourth speaker 204d, and the fifth speaker 204c. In some embodiments, the therapy device 1902 includes a same number of microphones 202 as speakers 204. For example, the therapy device 1902 may include a first microphone 202a, a second microphone 202b, a third microphone 202c, a fourth microphone 202d, and a fifth microphone 202e. In some embodiments, each of the microphones 202a-202e are positioned proximate a corresponding one of the speakers 204a-204c.

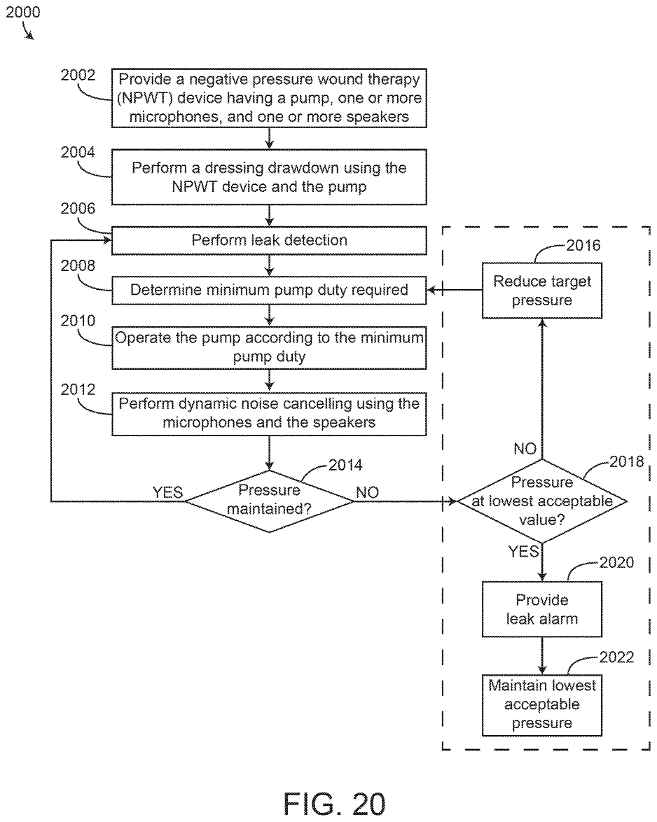

Referring to FIG. 20, a process 2000 for operating the therapy device 1902 is shown, according to some embodiments. The process 2000 includes steps 2002-2022 and can be performed by the therapy device 1902 and/or the controller 118 of the therapy device 1902. In some embodiments, the process 2000 is performed to provide active or dynamic noise cancellation by operating each of the speakers 204 independently or in combination with each other based on data obtained by each of the microphones 202 of the therapy device 1902.

Process 2000 includes providing a NPWT device having a pump, one or more microphones, and one or more speakers (step 2002), according to some embodiments. In some embodiments, step 2002 includes providing the therapy device 1902. In some embodiments, the NPWT device includes the microphones 202a-202e and the speakers 204a-204c.

Process 2000 includes performing a dressing drawdown using the NPWT device and the pump (step 2004), according to some embodiments. In some embodiments, the step 2004 is the same as or similar to the step 1204 of the process 1200 or the same as or similar to the step 904 of the process 900. In some embodiments, the step 2004 is performed by the controller 118 and the pump to draw the negative pressure at the dressing.

Process 2000 includes performing leak detection (step 2006), according to some embodiments. In some embodiments, the leak detection is performed by a controller of the NPWT device based on pressure readings obtained over time from a pressure sensor that is configured to measure pressure within the dressing. In some embodiments, the step 2006 is the same as or similar to the step 1206 of the process 1200 or the step 906 of the process 900.

Process 2000 includes determining a minimum pump duty that is required (e.g., to maintain the negative pressure at the dressing given any detected leaks) (step 2008), according to some embodiments. In some embodiments, step 2008 is the same as or similar to the step 1210 of the process 1200. In some embodiments, step 2008 is performed by the controller 118 of the therapy device 1902.

Process 2000 includes operating the pump according to the minimum pump duty (step 2010), according to some embodiments. In some embodiments, step 2010 includes generating control signals for the pump (e.g., the pneumatic pump 120) and providing the control signals to the pump to operate the pump according to the minimum pump duty. In some embodiments, step 2010 is performed by the controller 118.

Process 2000 includes performing dynamic noise cancelling using the microphones and the speakers (step 2012), according to some embodiments. In some embodiments, step 2012 includes obtaining data or sensor information from each of the microphones (e.g., the microphones 202 of the therapy device 1902) and using the data or sensor information to adjust an amplitude and/or phase (e.g., a frequency) of sound emitted by the speakers in order to provide active or dynamic noise cancellation. In some embodiments, each of the speakers are operated independently of each other based on data or sensor information from each of the corresponding microphones. In some embodiments, step 2012 includes adjusting a parameter or sound presentation property of each of the speakers based on sensor data obtained from the one or more microphones so that the sound emitted by the speakers destructively interferes with the sound emitted by the pump in each of a variety of directions.

Process 2000 includes determining if pressure is maintained (step 2014), according to some embodiments. In some embodiments, step 2014 is performed by the controller 118 based on pressure readings obtained from the pressure sensor of the NPWT device. If pressure is not maintained (step 2014, “NO”), process 2000 proceeds to step 2018. If pressure is maintained (step 2014, “YES”), process 2000 returns to step 2006.

Process 2000 includes determining if pressure is at a lowest acceptable value (step 2018) in response to determining that pressure is not maintained (step 2014, “NO”), according to some embodiments. In some embodiments, step 2018 includes comparing a current pressure setpoint to a lowest acceptable pressure setpoint. In response to the current pressure setpoint being greater than the lowest acceptable pressure setpoint (step 2018, “NO”) process 2000 proceeds to step 2016. In response to the current pressure setpoint being equal to or substantially equal to the lowest acceptable pressure setpoint (step 2018, “YES”), process 2000 proceeds to step 2020.

Process 2000 includes reducing target pressure (step 2016) in response to the target pressure not being at a lowest acceptable value (step 2018, “NO”), according to some embodiments. In some embodiments, step 2016 is performed by the controller 118. In some embodiments, step 2016 includes reducing the target pressure by a predetermined amount (e.g., reducing a magnitude of the negative pressure). In some embodiments, in response to performing step 2016, process 2000 returns to step 2008.

Process 2000 includes providing a leak alarm in response to the pressure being at a lowest acceptable value (step 2018, “YES”) and in response to the pressure not being maintained (step 2014, “NO”) (step 2020), according to some embodiments. In some embodiments, step 2020 includes operating an alert device, display screen, user interface, etc., to provide a leak alarm to a user, caregiver, clinician, etc. In response to providing the leak alarm, process 2000 proceeds to step 2022.

Process 2000 includes maintaining a lowest acceptable pressure (step 2022), according to some embodiments. In some embodiments, step 2022 is performed to maintain the lowest acceptable pressure to provide a minimal amount of negative pressure until a caregiver addresses the leak that occurs at the dressing.

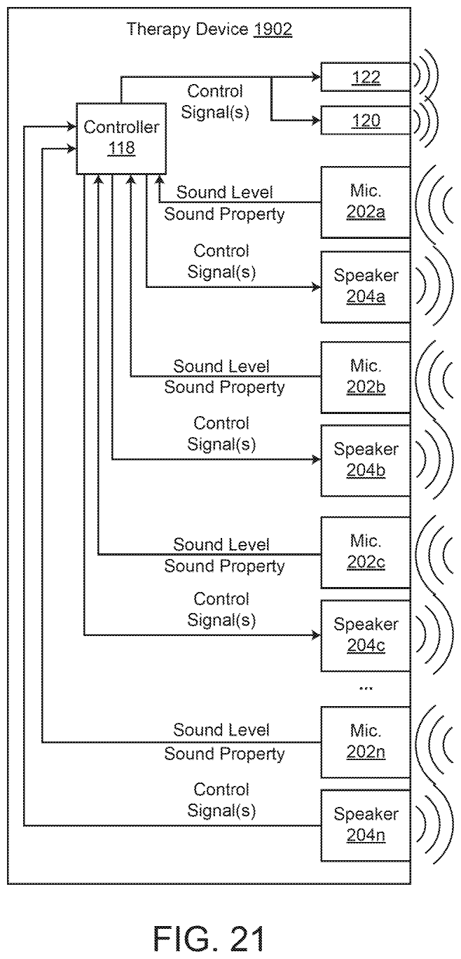

Referring to FIG. 21, the therapy device 1902 includes the controller 118, the instillation pump 122, and the pneumatic pump 120. The therapy device 1902 also includes the microphone 202a and the speaker 204a, the microphone 202b and the speaker 204b, the microphone 202c and the speaker 204c, etc., and a microphone 202n and a speaker 202n. The therapy device 1902 can include any number of microphones 202 and speakers 204, according to some embodiments. In some embodiments, the controller 118 is configured to obtain the sound levels and one or more sound properties from each of the microphones 202a . . . 202n and use the sound levels (e.g., amplitude) and the one or more sound properties (e.g., frequency) to determine control signals for the speakers 204a . . . 204n. In some embodiments, the control signals for the speakers 204a . . . 204n cause the speakers to operate to provide a sound having a same amplitude and an adjusted frequency or phase (e.g., anti-noise relative to the sound or noise measured at each of the microphones 202a . . . 202n) so that the speakers 204a . . . 204n provide active noise cancelling. In some embodiments, each of the speakers 204a . . . 204n are configured to provide noise cancelling (e.g., anti-noise) for the sound measured by the corresponding microphones 202a . . . 202n. For example, the microphone 202a can be configured to measure the sound level and the sound property of sound at a first location about the therapy device, and the controller 118 uses the sound level and the sound property obtained by the microphone 202a to determine control signals for the speaker 204a so that the speaker 204a operates to provide anti-noise for the sound at the microphone 202a. Similarly, the microphones 202b . . . 202n can provide sound levels and properties that are used by the corresponding speakers 204b . . . 204n in order to provide noise cancellation. In some embodiments, the controller 118 can also be configured to operate or adjust operation of the pneumatic pump 120 and/or the instillation pump 122 in combination with operation of the speakers 204a . . . 204n to reduce sound output or sound level of the therapy device 1902. Advantageously, reducing the noise output of the therapy device 1902 can be beneficial in a variety of cases, such as when the therapy device 1902 is used in public, during nighttime, etc.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A negative pressure wound therapy (NPWT) device comprising:
a pneumatic pump operable to draw a negative pressure at a wound dressing;
a single microphone configured to measure a sound level of an environment surrounding the NPWT device; and
a controller configured to:
operate the pneumatic pump to draw the negative pressure at the wound dressing;
obtain the sound level of the environment surrounding the NPWT device;
adjust operation of the pneumatic pump based on the sound level of the environment.

2. The NPWT device of claim 1, wherein the controller is configured to:
adjust a duty cycle of the pneumatic pump to reduce sound emitted by the pneumatic pump in response to the sound level of the environment exceeding a threshold.

3. (canceled)

4. The NPWT device of claim 1, further comprising a pressure sensor, wherein the controller is configured to determine if the negative pressure is maintained at the wound dressing based on information received from the pressure sensor.

5. The NPWT device of claim 4, wherein the controller is configured to, in response to the negative pressure not being maintained at the wound dressing:
determine if the negative pressure is at a lowest acceptable value;
in response to the negative pressure not being at the lowest acceptable value:
reduce a magnitude of the negative pressure by adjusting operation of the pump; and
obtain the sound level of the environment surrounding the NPWT device;
in response to the negative pressure being above the lowest acceptable value:
operate a display of the NPWT device to provide a leak alarm; and
operate the pneumatic pump to maintain the negative pressure at the lowest acceptable value.

6. The NPWT device of claim 4, wherein the controller is configured to, in response to the negative pressure being maintained at the wound dressing:
obtain the sound level of the environment surrounding the NPWT device; and
adjust operation of the pneumatic pump based on the sound level of the environment to maintain the sound level of the environment below a threshold.

7. (canceled)

8. The NPWT device of claim 1, wherein the controller is configured to:
operate the pneumatic pump to provide the negative pressure at a reduced magnitude in response to the sound level of the environment exceeding a threshold and a pump duty cycle of the pneumatic pump being at a lowest acceptable value.

9. A negative pressure wound therapy (NPWT) device comprising:
a pneumatic pump operable to draw a negative pressure at a wound dressing;
a single microphone configured to measure a sound level of an environment surrounding the NPWT device;
a single speaker configured to emit a sound for noise cancellation; and
a controller configured to:
obtain the sound level of the environment surrounding the NPWT device as the pneumatic pump operates to draw the negative pressure;
adjust a duty cycle of the pneumatic pump in response to the sound level of the environment exceeding a threshold;
obtain the sound level of the environment surrounding the NPWT device as the pneumatic pump operates according to the adjusted duty cycle;
in response to the sound level of the environment surrounding the NPWT device exceeding the threshold as the pneumatic pump operates according to the adjusted duty cycle:
operate the single speaker to provide a sound wave having a frequency to provide destructive interference to sound waves emitted into the environment by the pneumatic pump to reduce the sound level of the environment.

10. The NPWT device of claim 9, wherein the controller is configured to:
select a profile for the speaker from a plurality of predetermined profiles based on at least one of the adjusted duty cycle of the pneumatic pump or the sound level of the environment;
operate the speaker according to the profile to emit the sound wave for noise cancellation.

11. The NPWT device of claim 9, wherein the single microphone is configured to measure both a sound level of the environment, and a characteristic of sound waves in the environment, wherein the controller is configured to determine the frequency for the sound wave provided by the single speaker based on the characteristic of the sound wave in the environment, and wherein the characteristic comprises at least one of an amplitude or a frequency of sound waves in the environment of the NPWT device.

12. (canceled)

13. (canceled)

14. (canceled)

15. (canceled)

16. The NPWT device of claim 9, wherein the controller is configured to adjust operation of the pneumatic pump to provide the negative pressure having a reduced magnitude in response to the sound level of the environment exceeding the threshold amount as the single speaker is operated to provide the sound wave.

17. A negative pressure wound therapy (NPWT) device comprising:
a pneumatic pump operable to draw a negative pressure at a wound dressing;
at least one microphone configured to measure a sound level of an environment surrounding the NPWT device;
a plurality of speakers configured to emit sound for noise cancellation in a plurality of directions; and
a controller configured to:
obtain the sound level of the environment surrounding the NPWT device as the pneumatic pump operates to draw the negative pressure;
adjust a duty cycle of the pneumatic pump in response to the sound level of the environment exceeding a threshold;
obtain the sound level of the environment surrounding the NPWT device as the pneumatic pump operates according to the adjusted duty cycle;
in response to the sound level of the environment surrounding the NPWT device exceeding the threshold as the pneumatic pump operates according to the adjusted duty cycle:
operate the plurality of speakers to provide sound waves in the plurality of directions, the sound waves having frequencies to provide destructive interference to sound waves emitted into the environment by the pneumatic pump to reduce the sound level of the environment.

18. The NPWT device of claim 17, wherein the controller is configured to:
select a profile for each of the plurality of speakers from a plurality of predetermined profiles based on at least one of the adjusted duty cycle of the pneumatic pump or the sound level of the environment;
operate the plurality of speakers according to each of the profiles to emit the sound waves for noise cancellation.

19. The NPWT device of claim 17, wherein the single microphone is configured to measure both a sound level of the environment, and a characteristic of sound waves in the environment, wherein the controller is configured to determine the frequency for the sound wave provided by the single speaker based on the characteristic of the sound wave in the environment, and wherein the characteristic comprises at least one of an amplitude or a frequency of sound waves in the environment of the NPWT device.

20. (canceled)

21. The NPWT device of claim 17, wherein at least one of the plurality of speakers is positioned proximate a location on the NPWT device where sound waves emitted by the pneumatic device are propagated into the environment of the NPWT device.

22. The NPWT device of claim 21, wherein the pneumatic pump is positioned within a cavity of the NPWT device, the cavity comprising sound absorbing material and an opening, wherein the sound absorbing material is configured to absorb sound emitted by the pneumatic pump as the pneumatic pump operates, and the pneumatic pump is configured to emit sound through the opening to the environment, wherein the opening is the location of the NPWT device where sound waves emitted by the pneumatic device are propagated into the environment of the NPWT device.

23. (canceled)

24. The NPWT device of claim 17, wherein the controller is configured to adjust operation of the pneumatic pump to provide the negative pressure having a reduced magnitude in response to the sound level of the environment exceeding the threshold amount as the single speaker is operated to provide the sound wave.

25. A negative pressure wound therapy (NPWT) device comprising:
a pneumatic pump operable to draw a negative pressure at a wound dressing;
a plurality of microphones each configured to measure a sound level and a characteristic of an environment surrounding the NPWT device in a separate direction;
a plurality of speakers each configured to emit sound for noise cancellation in the separate directions; and
a controller configured to:
obtain the plurality of sound levels and the plurality of characteristic of the environment in each of the different directions as the pneumatic pump operates to draw the negative pressure;
in response to any of the sound levels of the environment surrounding the NPWT device exceeding a threshold as the pneumatic pump operates:
operate at least one of the plurality of speakers to provide sound waves in the plurality of directions, the sound waves having frequencies to provide destructive interference to sound waves emitted into the environment by the pneumatic pump to reduce the sound level of the environment in any of the plurality of directions.

26. The NPWT device of claim 25, wherein the controller is configured to determine the frequencies of the sound waves emitted by the plurality of speakers in each of the separate directions based on a corresponding one of the characteristics measured by one of the plurality of microphones.

27. The NPWT device of claim 25, wherein the controller is configured to operate each of the plurality of speakers independently in real-time based on the characteristics and sound level of sound in the different directions to provide active noise cancelling in each of the different directions.

28. The NPWT device of claim 25, wherein each of the plurality of speakers are positioned proximate a corresponding one of the plurality of microphones.
